(19) United States
(12) Patent Application Publication
(10) Pub. No.: US 2026/0191613 A1
(43) Pub. Date: Jul. 9, 2026

(54) PLUG AND PLAY CONNECTION

(71) Applicant: VIRTUALISURG, Paris (FR)

(72) Inventor: Tom LORENT BOURDO, Paris (FR)

(73) Assignee: VIRTUALISURG, Paris (FR)

(21) Appl. No.: 18/865,433

(22) PCT Filed: May 15, 2023
(86) PCT No.: PCT/FR2023/050697

§ 371 Date: Nov. 13, 2024

(30) Foreign Application Priority Data
May 13, 2022 (FR) FR2204583

(51) Int. Cl. A61B 34/00; A61B 34/10; A61B 34/30

(52) U.S. Cl. CPC A61B 34/76; A61B 34/10; A61B 34/30; A61B 2034/104

(57) ABSTRACT

A surgical training platform configured to interface a haptic controller with a surgical simulation tool. The platform includes a control system, at least one surgical simulation tool having at least one free end extending along a first axis, at least one haptic controller including a connection system configured to mechanically and electrically connect, reversibly, the at least one surgical simulation tool. The control system includes a system for recognising each surgical simulation tool configured to obtain identification information specific to the surgical simulation tool connected to the haptic controller, and to communicate the identification information to the control system in such a way that the control system recognises each surgical simulation tool connected to the haptic controller.

### FIELD OF THE INVENTION

The present invention relates to a system for connecting a surgical simulation tool to a haptic controller in a surgical training platform. The present invention is therefore in the field of educational and teaching tools, methods and equipment. More particularly, the invention relates to a connection system for a surgical procedure training kit, intended to train surgeons.

### PRIOR ART

To date, most surgical training is performed in real conditions, on patients, by way of surgical mentoring. This method requires significant human resources, has high hardware constraints and may generate significant stress for the student who may have difficulties in concentrating and/or remembering.

Alternatives exist, such as for example the Pelvitrainer EoSim SurgTrac® or certain sessions on animals. However, these training sessions/methods are only accessible to a small number of surgical interns and have a certain number of obvious limitations: the Pelvitrainer is a simple box wherein trocars and a camera are inserted with the possibility of practising stitches on inert materials such as foam. The animal model has obvious problems in terms of training quality because the anatomical similarities/correlations with humans are limited. The animal model also poses ethical problems.

The aim of the present invention is therefore to provide a safe, practical, accurate, realistic, easy to use and easily available training device, making it possible to give any surgical student an opportunity to train in a safe environment without any risk of injuring themselves, a patient or an animal. Furthermore, the user must be able to connect a surgical simulation tool to the platform easily and be able to manipulate the latter with no fear of pulling it out during its simulation. In addition, the user must be able to change the tools compatible with the simulation as they so wish, without hindrance, and smoothly.

### SUMMARY

Therefore, this invention relates to a surgical training platform configured to interface a haptic controller with a surgical simulation tool comprising:
a control system,
at least one surgical simulation tool having at least one free end extending along a first axis A₁,
at least one haptic controller including a connection system configured to mechanically and electrically connect, reversibly, the at least one surgical simulation tool,
   the control system further including a system for recognising each surgical simulation tool configured to obtain identification information specific to the surgical simulation tool connected to the haptic controller, and to communicate the identification information to the control system in such a way that the control system recognises each surgical simulation tool connected to the haptic controller.
   The invention is characterised in that the connection system comprises at least one connector extending along a second axis A₂, the at least one connector comprising a first coupling element complementary to a corresponding coupling element of the free end of each surgical simulation tool, the coupling elements of the connector and of the free end of each surgical simulation tool are configured to coaxially cooperate by alignment of the axes A₁ and A₂, in such a way that the connection between the haptic controller and each surgical simulation tool is carried out axially, once connected to one another, each movement of the surgical simulation tool induces a corresponding movement of the movable haptic controller.

Thus, the solution makes it possible to achieve the aforementioned objective. In particular, the platform according to the present invention makes a robust mechanical and electrical connection possible between the tool and the haptic controller, while making it possible for the user to change tools without exiting the simulation thereof.

The platform according to the invention may comprise one or more of the following features, considered separately from one another or in combination with one another:
the connection system of the haptic controller makes it possible to connect at least two different surgical simulation tools,
the at least one connector is configured to cooperate with the free end of the surgical simulation tool,
the at least one connector is a locking connector of the key-lock type configured to cooperate with the free end of the surgical simulation tool,
the connection between the haptic controller and the free end of the at least one surgical simulation tool ensures a colinearity constraint along the first axis A₁ of the at least one connector,
the connection system comprises a first connector attached to the haptic controller and a second connector attached to the free end of the surgical simulation tool, the two connectors being configured to cooperate with one another,
the haptic controller comprises a movable arm, the movable arm having a free end intended to cooperate with the connection system,
the connection system makes it possible to pass electric current between the haptic controller and the connected surgical simulation tool, in such a way as to supply the surgical simulation tool with power,
the connection system makes it possible to pass electric current between the control system and the connected surgical simulation tool, in such a way as to supply the surgical simulation tool with power,
each connector comprises a magnet in such a way that the connection is magnetised.

### BRIEF DESCRIPTION OF THE FIGURES

The invention will be better understood, and other aims, details, features and advantages thereof will become more apparent upon reading the following detailed description of embodiments of the invention, given purely by way of illustrative and non-limiting examples, with reference to the appended drawings, wherein:

FIG. 1 is a perspective overview of a disassembled platform according to the present invention,

FIG. 2 is a perspective overview of a disassembled platform according to the present invention, shown with two different surgical training tools each being able to be connected to the platform,

FIG. 3 is a view similar to FIG. 2 under a different angle,

FIG. 4A is a perspective view of an assembled training platform according to the invention, connected to a surgical training tool, including only one training module,

FIG. 4B is a perspective view of an assembled training platform according to the invention, including two training modules, connected to two surgical training tools,

FIG. 5 is a perspective view of a kit according to the present invention, manipulated by a user according to a first embodiment,

FIG. 6 is a perspective view of a kit according to the present invention, manipulated by a user according to a second embodiment,

FIG. 7 is an exploded view of a control module,

FIG. 8A is a perspective view of a calibration element associated with the display device according to the invention,

FIG. 8B is a perspective view of the calibration module,

FIG. 9A is a perspective view of a haptic controller connector according to the invention,

FIG. 9B is a perspective view of a surgical training tool connector according to the invention,

FIG. 10 is a perspective view of a surgical training tool,

FIG. 11 is a perspective view of one example of virtual environment according to the invention,

FIGS. 12 and 13 are detail views of a connection between a surgical simulation tool and the platform according to two different embodiments,

FIG. 14 is a perspective view of a haptic controller according to the invention, with all of the articulations.

### DETAILED DESCRIPTION

### Description of One Example of Surgical Simulation Platform

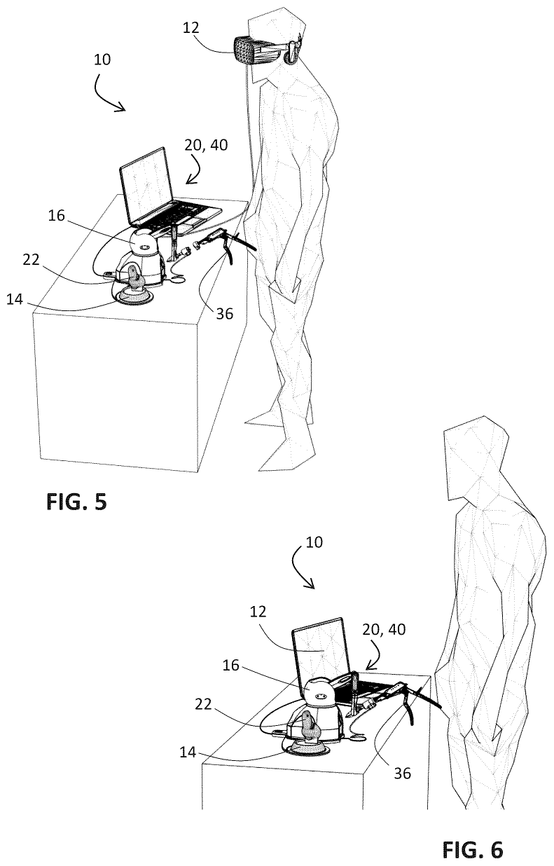

In the interest of clarity, one example of surgical simulation platform is detailed hereinafter, in order to place the connection according to the present invention, in a technical context.

[As can be seen in FIGS. 5 and 6, the present invention relates to a surgical training platform (for example modular) 10 configured to interface a virtual environment 100 comprising at least one movable virtual surgical element 102 (see FIG. 11). This virtual environment also comprises a virtual patient 104 and various decorative elements 106. Thus, a user manipulating the modular platform 10 interacts with the virtual environment 100 wherein all kinds of surgical operations are possible.

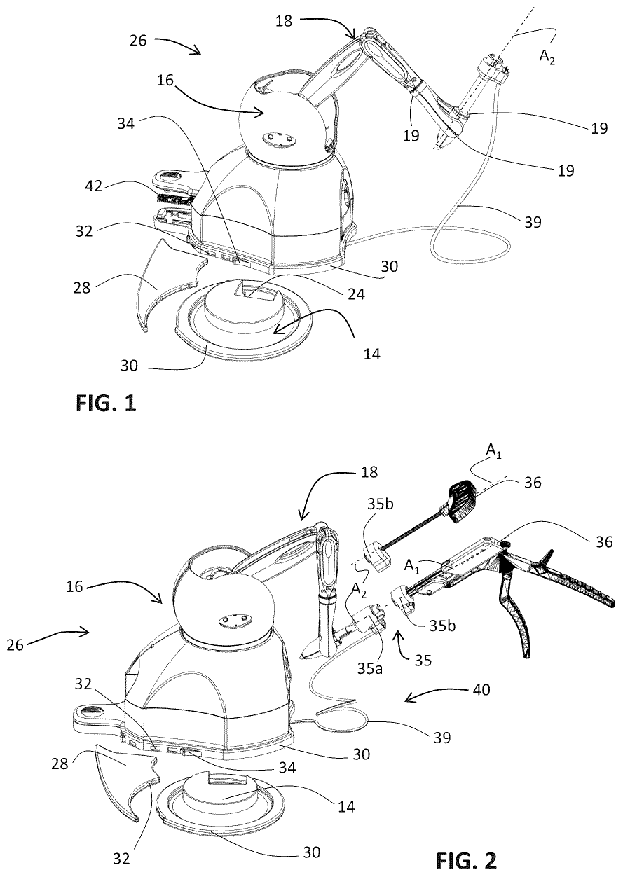

As can be seen in FIG. 1, the modular platform 10 according to the present invention comprises:
a virtual reality display device 12 configured to show/display, to the user, the virtual environment 100,
a calibration module 14 connected to the virtual display device 12,
at least one training module 16, each training module including a haptic controller 18,
possibly another optional module (not shown) that may ensure an ancillary function during the simulation,
a control system 20.

Within the scope of the present invention, the display device 12 creates the link between the various training modules 16 and the virtual environment 100. In the interest of simplification, only two types of modules will be considered in this description: the training 16 and calibration modules 14. However, all transpose to other functional modules. In this simplified example, the training module(s) 16 is/are the only element(s) manipulated by the user and the rendering of this manipulation can only be seen in the virtual environment 100. Each training module is an autonomous entity including a plurality of plastic parts assembled with one another. These parts may be 3D printed. All of these parts will be described throughout the present description. Thus, each movable virtual surgical element 102 and each virtual movement of each of these movable virtual surgical elements 102 present in the virtual environment 100 are made visible to the user through the display device 12.

More particularly, the display device 12 (that can be seen in FIGS. 5 and 6) may be an element fixed in space (such as a screen) or an element movable in space, for example, configured to be carried by the user during the surgical operation. The display device 12 may include a plurality of display devices, making it possible for a plurality of users to view the virtual environment 100. The various display devices may be movable or fixed. More precisely, as can be seen in FIGS. 5 and 6, the display system 12 may be a screen installed on a nearby surface or at a distance from the various modules 14, 16. In another embodiment, the display device 12 may be a virtual reality headset, that can be adjusted to the user and that may be able to provide an audio feedback. More particularly, it may concern an HP reverb® headset having two screens with a resolution of 2160×2160 pixels. Each screen has a display frequency of 90 Hz. The display device 12 is connected to the control system 20 preferably by a cable (for example a displayport or hdmi cable).

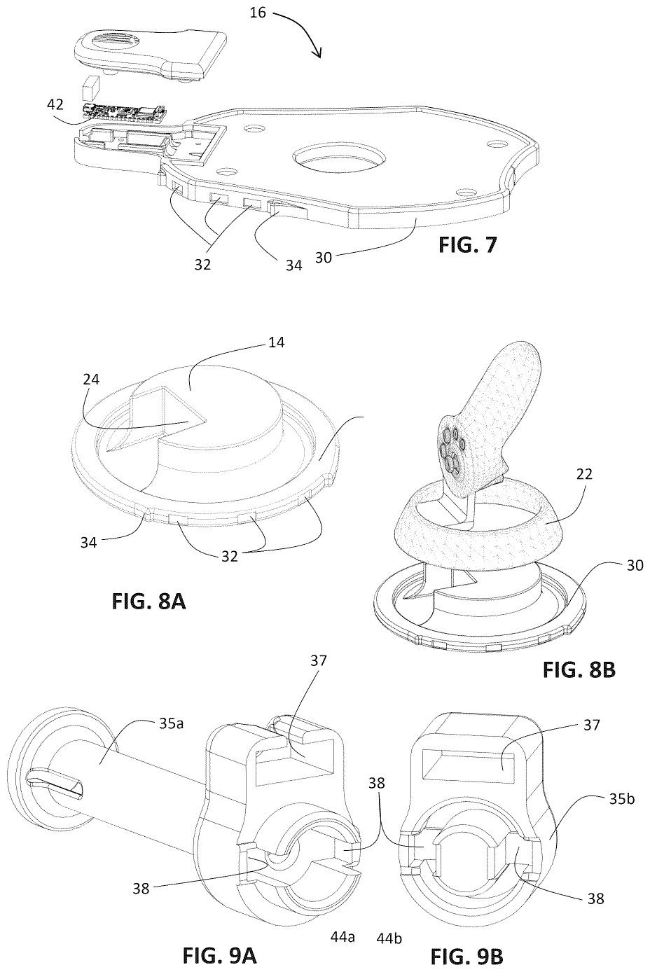

In a manner known per se, the display device 12 is associated with a movable calibration tool 22 (see FIG. 8B). The movable calibration tool 22 may take the form of a conventional controller such as for example illustrated in FIG. 8B but it may also take a different form.

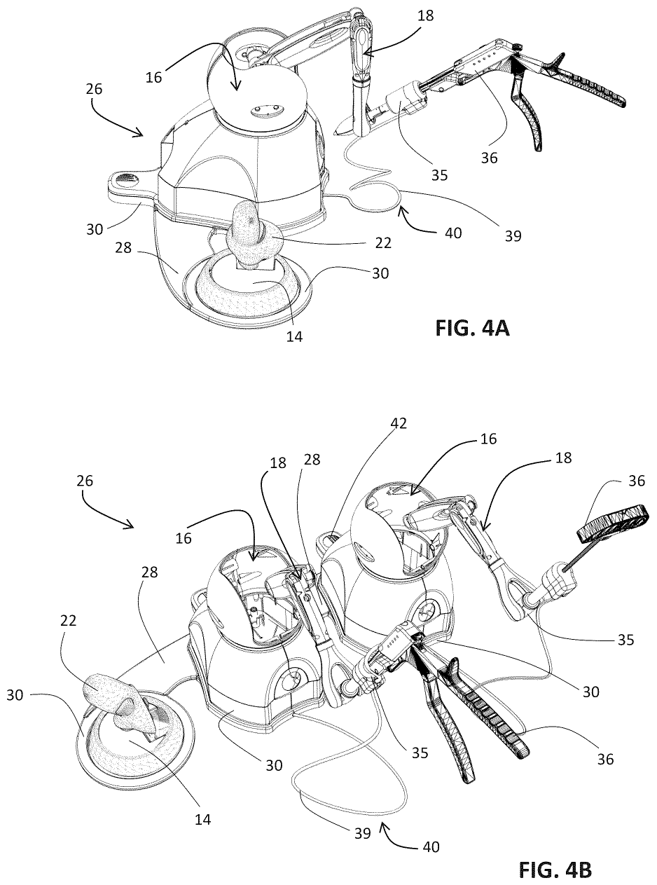

The calibration module 14 is an independent part shown in FIG. 8A. As can be seen in FIG. 8B, the calibration module 14 includes a footprint 24 complementary to the movable calibration tool 22. Thus, the calibration module 14 makes it possible to position the movable calibration tool 22 associated with the display device 12 at a known and fixed distance from the training module 16, in particular from the haptic controller 18 of the latter (see FIG. 4A). The calibration module 14 is preferably made of plastic. Preferably, it is 3D printed. In the same way as for the training modules 16, the calibration module 14 may include magnets, as will be explained in detail below. The calibration module 14 may also be provided with an electric connector for connecting an electronic circuit for identifying the calibration module 14 by the control system 20. For this, the same device including a voltage divider bridge as the one used for the key-lock connector of the haptic controller 18 is used, which will be described below.

As the haptic controller 18 makes it possible to know the position and the relative orientation of an object that is attached thereto (see below), the position and the orientation of this object is then obtained in relation to the movable calibration tool 22. In the case where the display device 12 is a movable device configured to be carried by the user, the calibration module 14 further makes it possible to locate the user in relation to the training module 16. Indeed, as the position of the movable calibration tool 22 in relation to the display device 12 is known, it is then possible to know the position and the orientation of the object connected to the haptic controller 18 in relation to the user who carries the display device 12 (see FIGS. 5 and 6).

Similarly, the various training modules 16 connected to one another or to the calibration module 14 may be positioned and located by the display device 12, given that once the various modules 14, 16 have been connected to one another, they are all at a fixed and known distance from the calibration module 14 and therefore from the movable calibration tool 22 (see FIGS. 4A and 4B). The possible identification of the various modules 16 through an electronic system (see below) may make it possible to know this distance in a “plug and play” manner. Thus, the movable calibration tool 22 associated with the display device 12 serves as a calibration reference for each training module, therefore for each haptic controller 18 and therefore for each of the physical elements manipulated by the user of the platform 10.]

In the present application, the notion of “plug and play” describes a simple action, that only implies a limited number of movements, preferably only one. A “plug and play” connection thus describes a connection that is carried out in only one movement.

An impact (sudden movement of the user or a manipulation error, for example) may result in an untimely displacement of the training module(s) 16 and therefore of the calibration module 14 that is connected thereto, in relation to the display system 12. This may lead to a calibration rupture between the virtual environment 100 and the position of the user. This may be avoided by using an electronic system including an accelerometer making it possible, on the one hand, to detect this type of untimely movements and, on the other hand, to adapt the digital positioning of the virtual environment 100 to the new position of the calibration module 14 with the movable calibration tool 22.

The various training modules 16 may be connected to one another, in such a way as to form a control console 26 (see FIG. 4B). The control console 26 thus comprises at least one training module 16 (see FIG. 2). More particularly, all of the modules 14, 16 are configured to be reversibly attached to one another in a known configuration (see FIGS. 4A and 4B). This makes the modularity of the platform 10 possible according to the invention.

The training module(s) 16 forming the control console 26 may either be connected directly to one another, or connected to one another by means of spacer modules 28 (see FIGS. 4A and 4B). The spacing modules 28 are made of plastic and are preferably produced by 3D printing, layer deposition or sintering. According to other embodiments, they may be manufactured by moulding or subsequently other methods. The spacer modules 28 are connection parts for creating a known spacer (therefore a positioning) between the various modules 14, 16 of the platform 10.

Each calibration 14 or training module 16 to this end comprises a base 30 having a specific shape (see FIG. 7). The shapes of the various bases 30 of the various modules 14, 16 are complementary to one another, in such a way as to obtain a stable and suitable interlocking of the various modules 14, 16, 28 with one another. The known aspect of the bases 30 of the various modules 14, 16, 28 makes it possible to easily determine the relative position of the modules 14, 16, 28 in space. Preferably, the calibration 14 and training modules 16 are connected to one another by means of spacer modules 28 in order to increase the stability of the control console and of the platform 10 in its entirety when the latter is assembled.

According to the embodiment shown in FIGS. 1, 2, 7 and 8A, the physical connection of the various modules 14, 16, 28 to one another is carried out by means of a magnetic interlocking system for easy interlocking of the various modules 14, 16, 28 with one another. More precisely, each base 30 of each module 14, 16, 28 includes at least one magnet 32 intended to cooperate with a corresponding magnet 32 of a base 30 of a complementary module 14, 16, 28, thus forming a magnetic connection point. Preferably, the magnets 32 are grouped in threes at each magnetic connection point. In the scenario where the modules of the control console 26 and the calibration module are connected to one another by spacer modules 28, the polarity of the magnets 32 is chosen so that the spacer modules 28 and the other modules (calibration modules 14 and training modules 16) attract. The presence of a magnetic interlocking system makes it possible to stabilise the interlocking between the various modules 14, 16, 28 and to limit the untimely releases in the event of clumsiness of the user or of involuntary jolts.

The physical connection may, furthermore, include an electronic connector 34 for the electronic communication between the various modules 14, 16, 28 and the control system 20 (see FIGS. 1, 2, 7 and 8A).

Optionally, in the case where the calibration modules 14 and the training modules 16 are connected to one another by means of spacer modules 28, each spacer module 28 may receive at each magnetic connection point, in addition, an electronic connector 34 intended to cooperate with an electronic connector of the base 30 of the calibration and/or training modules 16. The electronic communication between the various modules 14, 16, 28 is therefore ensured, that the calibration 14 and training modules 16 are connected to one another directly by means of a spacer module 28.

Each electronic connector 34 may be connected to a cable to connect the electronic connector of the corresponding connected module. These electronic connectors 34 may, for example, take the form of pin connectors on retractable springs/pins. In certain embodiments, each electronic connector 34 associated with a training module 16 includes for example, a voltage divider bridge generating a voltage specific to each training module 16. This makes it possible to identify the training module 16 by reading the voltage generated by the voltage divider bridge. In other embodiments, the electronic connector 34 forms part of a more complex electronic circuit able to engage a digital communication (for example meeting the “UART” standard).

Many technologies exist for identifying physical connection modules using electronic technologies, but they are not, however, used in a virtual reality context for surgical education.

In summary, this electronic connection makes it possible to:
identify modules 14, 16 by the control system 20, and/or
transmit displacement information of the haptic controller 18 (see below).

### Description of the Connection With the Surgical Simulation Tool

The control system 20 includes a microcontroller itself electrically connected, through the connectors 34 and potentially the spacer modules 28, to the calibration 14 and training modules 16. This connection will be detailed below.

In a first embodiment/operation the various calibration 14 and/or training modules 16 integrate a voltage divider system. The microcontroller then reads the voltage and is able to identify the module(s) 14, 16 that meet(s) this voltage. In an alternative embodiment/operation, the calibration 14 and/or training modules 16 each include an electronic card 42 for a digital communication with the microcontroller of the training module 16. They identify one another and are able to exchange information relating to an action of the user but also feedback to the user of the control system 20 (it can be envisaged for example a module that lights up red if an error is made in the manipulation).

As mentioned above and as can be seen in FIGS. 1, 2 and 3, each training module 16 includes a haptic controller 18.

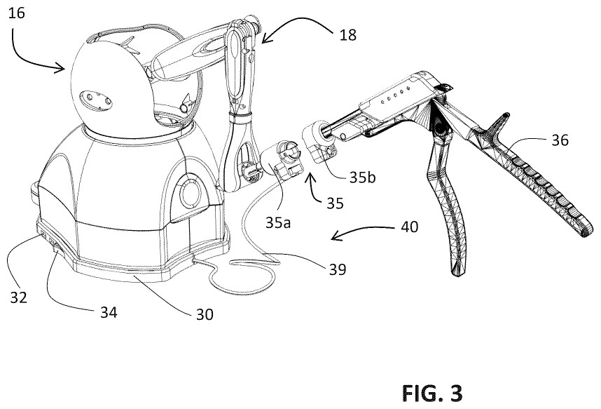

As can be seen in FIGS. 2 and 3. Each haptic controller 18 includes a connection system 35 configured to mechanically connect, reversibly, a surgical training tool or a surgical simulation tool 36.

More particularly, the surgical training platform according to the present invention is specifically configured to interface the haptic controller 18 with at least one surgical simulation tool 36.

To make the virtual reality surgical learning device proposed by the platform 10 according to the present invention more immersive and more realistic, it is interesting that the user can manipulate physical tools to control the simulation that is displayed in the display system 12. In a manner known per se, the closer these physical tools are to the original surgical tool, the more immersive the simulation.

This is why the present invention operates, as a kit, with a series of surgical simulation tools 36 (see FIG. 2). The surgical training kit thus formed (see FIGS. 5 and 6), therefore comprises a modular surgical training platform 16 an example of which is described in the present description and at least one surgical simulation tool 36 configured to be connected to the haptic controller 18 of said platform 10. The kit according to the present invention may include three types of surgical simulation tools 36:
the so-called “simple” tools shown to the user in the virtual reality 100,
the so-called “complex” tools shown to the user in the virtual reality 100, and
the tools not shown to the user in the virtual reality 100.

The complex surgical simulation tools 36 are complex electronic tools that integrate a microcontroller able to communicate directly with the control system 20.

The surgical training tools 36 shown to the user, simple or complex, are modified surgical tools or copies of the latter. The simulation permitted by the platform 10 according to the present invention, thus makes all or part of the physical actions to which these objects are subjected correspond with the behaviours of virtual twins in the virtual environment 100 displayed by the display device 12 (see FIGS. 5 and 6). The tools not shown make it possible to simulate sensations for example for organ palpation within a patient.

Each surgical simulation tool 36 having at least one free end extending along a first axis A₁.

As can be seen in the embodiment illustrated in FIGS. 2 and 3, the haptic controller 18 of each training module 16 comprises a swivelling-arm robot, the swivelling-arm robot having a free end intended to cooperate with the connection system 35.

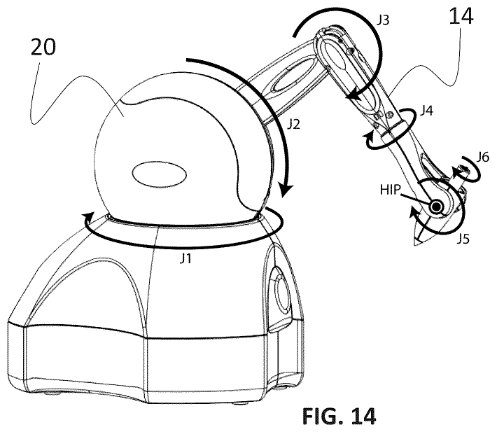

As shown in FIG. 14, the haptic controller 18 is movable according to at least six degrees of freedom obtained by means of various elbows and rotary parts cooperating with one another in such a way as form articulations J₁, J₂, J₃, J₄, J₅, J₆. More precisely, and as can be seen in FIG. 6, the first three articulations (distal articulations) can be activated by the user whereas the last three articulations (proximal articulations) are passive.

In order to maximise the realism of the simulation, the surgical simulation tool 36, once connected to the haptic controller 18, must have the point thereof (or the free end thereof) positioned where the haptic feedback would occur in reality, that is to say at the haptic point of the haptic arm. This haptic point is designated as “HIP” in FIG. 14. The haptic controller 18 simulates the force feedback related to the collision or to the interactions in the virtual world of the point (or of the end) of the surgical simulation tool 36 manipulated with an element of the virtual environment 100. This is the point on which the interactions and collisions are calculated in order to be able to simulate them without creating a shift or a haptic inconsistency, uncomfortable and disturbing, for the user. Taking into account this haptic point HIP, makes it possible to simulate the penetration of the body of a patient, for example by the needle of a syringe, simulating the constraint exerted by the body of the patient on the needle. It is therefore very important that the surgical simulation tool 36 and the haptic controller 18 are connected accurately and robustly.

The connection system 35 of the haptic controller 18 is universal, in the meaning that it makes it possible to connect at least two different surgical training tools 36 (see FIG. 2).

The connection system 35 comprises at least one connector 35a extending along a second axis A₂. This connector 35a comprises a first coupling element 44a complementary to a corresponding coupling element 44b of the free end of each surgical simulation tool 36.

More precisely the connection system 35 comprises at least one locking connector 35a of the key-lock type configured to be attached to the haptic controller 18 and to any surgical training tool 36 in such a way as to ensure the removable connection thereof (see FIGS. 3, 9A and 9B).

The coupling elements 44a, 44b of the connector 35a and of the free end of each surgical simulation tool 36 are configured to coaxially cooperate by alignment of the axes A₁ and A₂, in such a way that the connection 35 between the haptic controller 18 and each surgical simulation tool 36 is carried out axially.

Once connected to one another, each displacement of the surgical simulation tool 36 induces a corresponding movement of the movable haptic controller 18. Thus, the connectors 35a, 35b also make it possible to transmit the rotation along the axis from the end of the haptic controller 18 to the training module 16.

In one embodiment shown in FIG. 13, the at least one connector 35a is configured to cooperate with the free end of the surgical simulation tool 36.

On the embodiment shown in FIGS. 3, 4A and 4B, the connection system 35 comprises two connectors 35a, 35b, in particular two locking connectors 35a, 35b of the key-lock type: a first connector 35a attached to the haptic controller 18 and configured to removably cooperate with a corresponding second connector 35b attached to the surgical training tool 36. The two connectors 35a, 35b may both be reversibly attached, either to the haptic controller 18, or to the surgical training tool 36. The two connectors 35a, 35b, are obtained by 3D printing, either by layer deposition, or by sintering. In the example of FIGS. 9A and 9B, FIG. 9A shows the first connector 35a (here the lock) of the haptic controller 18 and FIG. 9B shows the second connector 35b (here the key) intended to cooperate with the surgical training tool 36. Attaching the first connector 35a on the haptic controller 18 may be done in several ways. In the case of the illustration 9A, a jack interface already originally present on the haptic controller 18 was used to attach it. In other embodiments, it may be envisaged to glue the first connector 35a or adapt to another haptic controller 18 creating a form of interface specific to the latter. Attaching the second connector 35b to the surgical training tool 36 is preferably done by gluing. This mainly concerns gluing at the distal rod of the surgical training tool 36. The 3D model of the second connector 35b is adapted by arranging, on the face that cannot be seen in FIG. 9B, a drill hole corresponding to the end of the distal rod 360 of the surgical training tool 36. Then the distal rod is glued in the second connector 35b by means, for example, of epoxy. This manufacturing method is not the only one that is implemented. According to alternative embodiments, 3D printing a reproduction of a surgical training tool 36 of which the model would contain the second connector 35b may also be envisaged.

The connection 35, of the key-lock type by alignment of the axes A₁ and A₂ ensures a colinearity constraint along the axis X of the connector 35a of the free end of the haptic controller 18. The two connectors 35a, 35b (and therefore the surgical training tool 36 and the haptic controller 18) are therefore additionally constrained in all directions.

In order to stabilise the reversible connection between the haptic controller 18 and the surgical training tool 36, the connection system 35 may include, on each side of the “key-lock” system, at least one magnet 38 (see FIGS. 9A, 9B). Preferably, the magnets 38 used, are cubic magnets having a magnetising force of 1.1 kg. Preferably, the magnets 38 used are neodymium (cubic) magnets. In an alternative embodiment, annular magnets 38 are used. The latter make it possible to pass a stainless steel rod inside of them and to avoid any impact of shearing forces, increasing the stability of the connection 35. This value makes both a solidity of the attachment as well as an easy disconnection of the surgical training tool 36 possible, in the desired view of creating a “plug and play” interface. Indeed, the magnets 38 ensure a contact stress between the two connectors 35a, 35b.

The connection 35 may further comprise a screw configured to cooperate along an axis normal to the axes A₁ and A₂ once aligned, in such a way as to be normal to the pivot of the haptic controller 18 (see FIG. 13). In an alternative embodiment, the connection 35 may be locked to avoid the rotation of the coupling elements 44a, 44b in relation to one another. This makes it possible to ensure a strong link between the connection 35 and the haptic controller 18.

Still in the interest of reinforcing the connection 35, in certain embodiments, the coupling elements 44a, 44b of the connector 35a and of the free end of each surgical simulation tool 36 include a combination of a hole intended to cooperate with a rod protruding into the tool to avoid any effect of shearing forces that would lead to an untimely disconnection of the coupling elements 44a, 44b.

The two connectors 35a, 35b are thus completely constrained in all directions, except the direction colinear to the axis A₂ of the end of the haptic controller 18. Magnetisation makes it possible to constrain/maintain the connection 35 also in this axial direction along A₁ and A₂. However, the rupture force of this constraint (and therefore rupture of the “key-lock” connection 35) is lower along the axis A₁-A₂ because the force of the magnets 38 is not very high. The result obtained is therefore that the two connectors 35a, 35b separate by pulling on the surgical training tool 36 more strongly than what is needed to cause the movement of the haptic controller 18. Thus, the haptic controller 18 needs to be maintained in order to succeed in disconnecting the surgical training tool 36. The presence of magnets 38 makes the connection and the disconnection between the surgical training tool and the haptic controller 18 easier. Indeed, the magnets 38 make a simple connection/disconnection movement (of the “plug and play” type) possible without screws or slides: the user approaches the surgical training tool 36 of the haptic controller 18 and the latter alone connects through the action of the magnets 38.

The connection system 35 of the key-lock type according to the present invention has three distinct and complementary functionalities:
it makes it possible to easily attach and “plug and play” the surgical training tool 36 using magnets 38,
it makes it possible to transmit the rotational movement along a central axis to the haptic controller 18,
in some cases, it makes it possible to electrically connect a surgical training tool 36 to the training module 16.

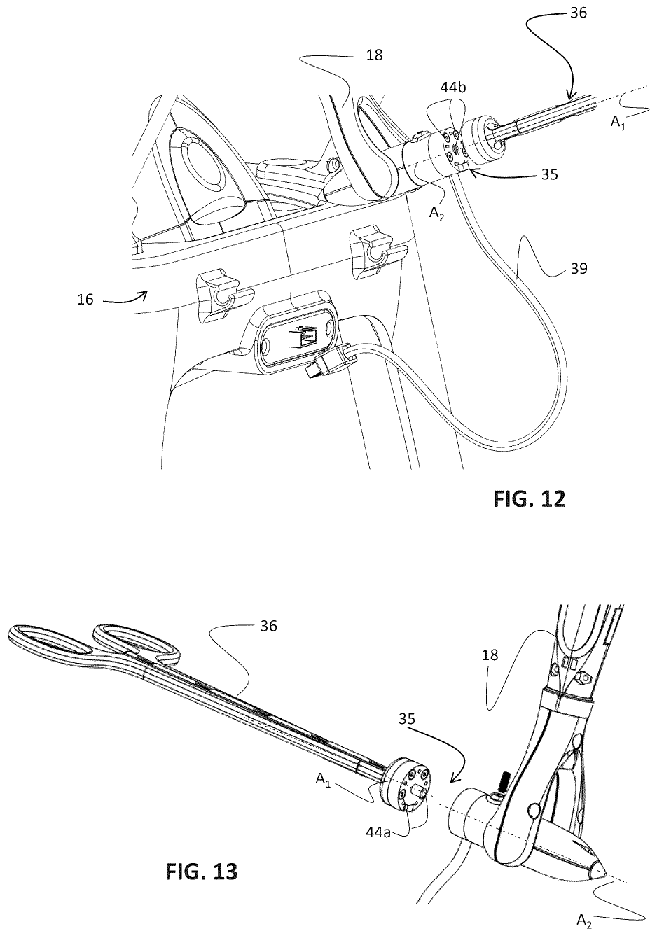

The connection system 35 thus has an electronic component. Each of the connectors 35a, 35b thus includes an opening, a groove or a recess 37 intended for inserting an electrical connector (not shown in FIGS. 9A, 9B). In a preferred embodiment, this electrical connector is a JST electrical connector having pins, retractable or not, but other types of electrical connectors can be used. Preferably, the male portion of the electrical connector is inserted into the opening 37 of the first connector 35a attached to the end of the haptic controller 18 arm. In this embodiment, the female portion of the electrical connector is inserted into the opening 37 of the second connector 35b of the surgical training tool 36. In another embodiment (FIG. 12-13), this concerns a “pogo pin” on springs placed circularly about the alignment axes A₁ and A₂. The coupling elements 44a, 44b of the connector 35a and of the free end of each surgical simulation tool 36 are aligned using a polarising device (for example a protuberance on the lock and opening/holes on the key of the tool). A locked connection 35 makes the alignment of the electrical pins possible and prevents involuntary contact between incompatible pins. Certain embodiments integrate a spring because the use of pins on springs makes the contact perfect between all of the pins and makes almost zero friction possible. The use of magnets 38 ensures a powerful contact between all of the pins.

As can be seen in FIGS. 2 and 3, a cable 39 connects the pins of the electrical connector to the control system 20. Preferably, this cable 39 is connected to the control module 16. According to a preferred embodiment, the cable 39 can be disconnected from the control module 16, as can be seen in FIG. 12. This connection between the cable 39 and the control module 16 is preferably carried out by means of a “plug and play” or “snap fit” magnetic connection.

Another advantage of the connection system 35 according to the present invention is the simplicity with which it is possible to change the surgical simulation tool 36 to the haptic controller 18. This simple and rapid change is necessary so as not to impede the learning of complex manipulations. Therefore, it is necessary to propose a “plug and play” device, as the present invention does.

Each haptic controller 18 is, furthermore, configured to measure each movement in space of the surgical training tool 36 once the latter has been connected to the haptic controller 18. Each haptic controller 18 is thus provided with at least one external rotational or translational sensor 19 attached on the various movable elements of the haptic controller 18 (see FIG. 1), in such a way as to capture the position and the three-dimensional orientation of any object connected to the haptic controller 18 of the training module 16.

Two movement categories can be distinguished:
those that may be qualified as external, common to all surgical training tools 36, and that correspond to the position and to the three-dimensional orientation of the surgical training tool in space, and
those that may be qualified as internal, specific to certain so-called complex surgical training tools 36, having an idle state and at least one activation state, such as pressing in a trigger or rotating an element and including an embedded electronic card.

These categories correspond to three types of surgical simulation tools 36 included in the surgical training kit according to the present invention.

The haptic controller 18 according to the present invention makes it possible to measure the external movements (movements in space) of each connected surgical simulation tool 36.

In the case of complex surgical simulation tools 36 and of certain so-called simple surgical simulation tools 36, the connection system 35 may also play one or more roles other than that of the identification of the surgical training tool 36 connected to the haptic controller 18:
it may make it possible to retrieve information about the displacement of elements specific to the tool, such as the action of a trigger, for example. Moreover, and/or
it may make it possible to supply the internal electronics of the surgical training tool 36 connected to the haptic controller 18,
it may make electronic communication possible between the surgical training tool 36 and the training module 16.

However, as mentioned above, the complex surgical simulation tools 36 have a microcontroller able to communicate directly with the control system 20, and therefore this means of communication is preferred. In this case, the connection to the haptic controller 18 through the connection system 35 is above all physically and mechanically useful. The advantage that this connection system 35 has for the complex tools is that, by connecting the tool it can be detected whether or not the tool is plugged in.

As regards the power supply of the connected tool 36, according to the embodiments, the connection system 35 makes it possible to pass electric current between the haptic controller 18 or the control system 20 directly and the connected surgical simulation tool 36, in such a way as to supply it with power. In the second embodiment, the haptic controller 18 is bypassed (as regards the power supply) and the power supply does not pass through it.

As the modularity of the platform 10 according to the example detailed above makes it possible to connect a plurality of training modules 16 to one another, to the calibration module 14 and to the control system 20, the platform 10 thus makes it possible to determine the positioning in space of a plurality of surgical training tools 36 connected to various haptic controllers 18. If the control console 26 includes a plurality of training modules 16, the platform 10 makes it possible to determine the positioning of a plurality of surgical training tools 36 simultaneously, as soon as the latter are connected to a haptic controller 18.

The control system 20 of the platform 10 according to the invention further includes a system for recognising 40 each surgical simulation tool 36. More precisely, the recognition system 40 of the control system 20 is configured to obtain identification information specific to each surgical simulation tool 36 connected to the haptic controller 18. The recognition system 40 is configured to communicate the identification information to the control system 20 in such a way that the control system 20 recognises each surgical simulation tool 36 connected to the haptic controller 18.

The recognition system 40 of the surgical training tool 36 is thus configured to:
read the voltage from a voltage divider bridge specific to each surgical training tool 36,
communicate with the control system 20, in such a way that the control system 20 recognises each surgical training tool 36 connected to the haptic controller 18.
In an alternative embodiment, the recognition system 40 of the surgical training tool 36 is thus configured to read an electronic identification chip located inside the surgical simulation tool 36, in particular the so-called simple tools.

Indeed, it is necessary to identify each surgical simulation tool 36 that is connected to the haptic controller 18 to make it possible for the control system 20 to generate, if applicable, a corresponding movable virtual surgical element 102 in the virtual space 100. In any case, it is necessary to have an identification so that the control system 20 can adapt the haptic response to the tool used.

However, in the case where the control system executes highly directional software indicating to a user which surgical simulation tool to use, this identification is not necessary because the simulation only operates with only one predetermined tool or a plurality of tools 36 in a predefined order.

According to the surgical training tool 36 considered, the platform 10 uses a wireless connection and/or an electrical connection to identify the connected surgical tool (see FIG. 3).

In the case of a simple surgical simulation tool 36, the recognition system 40 comprises a microcontroller 42 preferably located in the base 30 of the training module 16, as can be seen in FIG. 7. The microcontroller 42 is connected to the surgical simulation tool 36 by means of the connection system 35, by the cable 39. The recognition system 40, in the case of a simple surgical training tool further comprises, at the connection system 35 between the tool 36 and the haptic controller 18, an electronic device such as a voltage divider bridge in order to make it possible to recognise the tool 36.

In the case of a complex surgical training tool, the recognition system 40 of the control system 20 retrieves and analyses the information from the microcontroller of the complex surgical training tool 36. In this case, the wireless communication is sufficient for the identification.

The control system 20 of the platform 10 is configured to:
identify the various modules 14, 16, 28 connected to one another,
receive and analyse the data related to the movement(s) of each surgical training tool 36 connected to a haptic controller 18,
generate the virtual environment 100,
interface, if applicable, each movable virtual surgical element 102 of the virtual environment 100 with a corresponding real element,
generate a specific haptic feedback in relation with the connected tool, the movements of the user (therefore of the haptic controller 18) and the virtual reality 100.

The control system 20 thus generates, for the tools requiring it, a virtual image of each surgical simulation tool 36 connected to the haptic controller 18.

As already mentioned above, the virtual environment 100 also includes decorative elements 106 that cannot be moved and/or manipulated. This may for example concern an endoscopy screen 108 or a lamp that can be virtually manipulated by the user with, for example a click on a button to switch them on. These decorative elements 106 do not have corresponding real elements.

Based on the information received from the recognition system 40 and the information collected at the haptic controller 18, the control system 20 is configured to convert/reproduce each movement in space of each surgical simulation tool 36 connected to a haptic controller 18 of the control console 26 into a corresponding virtual movement of the virtual image 102 thereof in the virtual environment 100.

In the particular case of a connection with a simple surgical simulation tool 36, the control system 20 includes:
a measuring unit (or microcontroller 42) configured to:
   identify the tools 36 and/or the modules 14, 16 connected,
   collect specific movement (or internal movement) data of the connected surgical training tool 36,
a central processing unit configured to:
generate the virtual environment 100,
receive and analyse the data related to the movement(s) of each connected surgical simulation tool 36,
interface each virtual surgical tool 102 of the virtual environment 100 with a corresponding real element.

In this particular case, as illustrated in FIGS. 1 and 7, the measuring unit (or microcontroller 42) forms part of the training module 16.

The control system 20 is further configured, as mentioned above, to generate a return signal (or haptic signal) making it possible for the haptic controller 18 to in turn generate a corresponding haptic signal, depending on what happens in the virtual environment 100. Thus, the control system 20 leads the haptic controller 18 to generate a specific haptic feedback when the virtual tool 102 corresponding to the surgical training tool 36 manipulated by the user comes into contact with another virtual tool 102 or another virtual element such as a decorative element 106, of the virtual environment 100. This makes it possible to accentuate the immersive aspect of the simulation and to give a greater sense of reality; the interactions that can be seen in the virtual environment 100 are also felt by the user.

In the present application, the notion of “haptic signal” is understood as a signal actively generated by the platform 10 according to the present invention. It should be differentiated from the notion of “tactile feedback” that is a simple passive feedback, automatically generated by the human body in response to the manipulation of animate or inanimate objects.

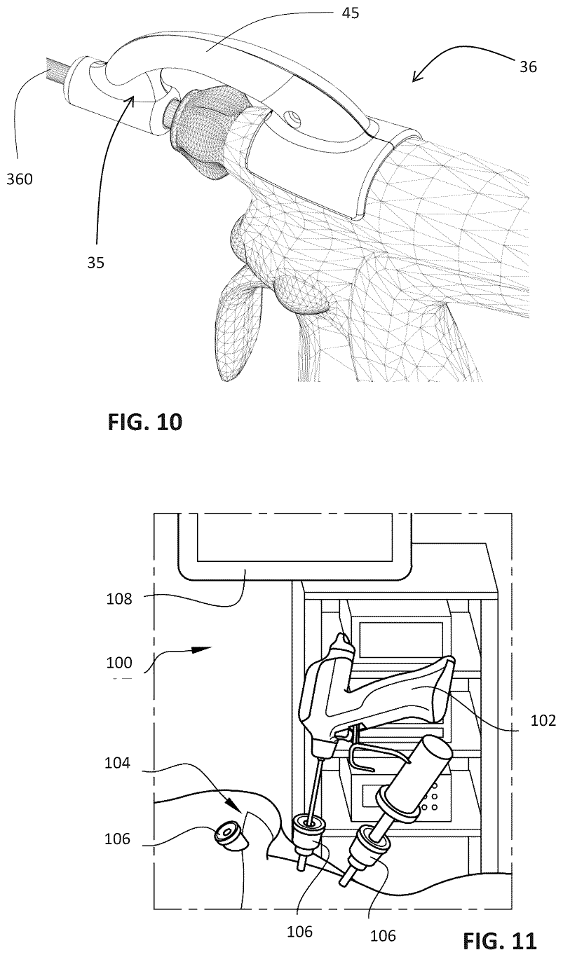

Some complex surgical simulation tools 36, such as for example that shown in FIG. 10, have a rotary distal rod 360. These tools 36 thus have a wheel for rotating the distal rod 360 and therefore the axis thereof. When this distal rod 360 is connected to the connection system 35, it is then impossible, for the control system 20 to measure/determine both the position in space of the surgical simulation tool 36 and the specific rotation of the distal rod 360: indeed the general rotation of the surgical simulation tool 36 needs to be transmitted in relation to the axis of the haptic controller 18 so that its virtual twin (virtual tool 102) can be oriented similarly in the virtual environment 100 without losing the specific rotation of the distal rod 360 induced by the operation of the surgical simulation tool 36.

To solve this problem, the connection system 35 has a particular embodiment with an arch part 45. The arch part 45, as shown in FIG. 10 makes it possible to freely rotate a wheel for orientating the rod on the surgical training tool 36 without in as much losing the information of the orientation of the tool 36 itself. The arch part 45 is attached on the gripping body of the tool 36 on the one hand and on the distal rod 360 secured to the second connector 35b on the other hand. The distal rod 360 is cut in such a way that the portion under the arch part 45 can be freely subjected to rotation without consequence on the rotation at the key-lock mechanism of the connection system 35, at the end of the haptic controller 18.

The arch part 45 is preferably printed using a layer deposition 3D printer, but any other plastic manufacturing method may be used such as for example laser sintering. The arch part 45 is preferably designed in two portions to be able to be easily removable, the two portions are assembled by means of screws.

Thus, it is observed that the platform 10 according to the invention is built around the training module(s) 16. Each training module 16 is thus a central element located at the convergence of the various elements of the platform 10 according to the present invention. Each training module 16 is organised, as already mentioned, around a base 30 that makes it possible to attach the various elements:
the microcontroller 42 and the connection cable 39 thereof to a connection system 35 intended to connect the surgical training tool 36 to the control system 20,
a haptic controller 18 including a robot for acquiring three-dimensional movement by polar coordinate system,
magnets 34, 38, and, possibly
one or more electrical connectors (for example retractable pin connectors as seen above).

These various elements together make it possible, once connected to the virtual reality display device 12, by means of the calibration module 14, to connect, within a context of surgical intervention simulation, the manipulation of physical surgical objects to their virtual twins in a virtual reality simulation. The platform 10 makes a simple but mechanically and electrically robust connection possible enabling a user to use the platform 10 in complete safety and with peace of mind, without having to worry about how the surgical simulation tool 36 is manipulated and without being handicapped by a heavy and/or cumbersome connection system.

1-10. (canceled)

11. A surgical training platform configured to interface a haptic controller with a surgical simulation tool comprising:
a control system,
at least one surgical simulation tool having at least one free end extending along a first axis A₁,
at least one haptic controller including a connection system configured to mechanically and electrically connect, reversibly, the at least one surgical simulation tool, and
the control system further including a system for recognising each surgical simulation tool configured to obtain identification information specific to the surgical simulation tool connected to the haptic controller, and to communicate the identification information to the control system in such a way that the control system recognises each surgical simulation tool connected to the haptic controller,
wherein:
the connection system comprises at least one connector extending along a second axis A₂, the at least one connector comprising a first coupling element complementary to a corresponding coupling element of the free end of each surgical simulation tool,
the coupling elements of the connector and of the free end of each surgical simulation tool are configured to coaxially cooperate by alignment of the axes A₁ and A₂, in such a way that the connection between the haptic controller and each surgical simulation tool is carried out axially, once connected to one another, each displacement of the surgical simulation tool induces a corresponding movement of the movable haptic controller.

12. The surgical training platform according to claim 11, wherein the connection system of the haptic controller makes it possible to connect at least two different surgical simulation tools.

13. The surgical training platform according to claim 11, wherein the at least one connector is configured to cooperate with the free end of the surgical simulation tool.

14. The surgical training platform according to claim 13, wherein the at least one connector is a locking connector of the key-lock type configured to cooperate with the free end of the surgical simulation tool.

15. The surgical training platform according to claim 11, wherein the connection between the haptic controller and the free end of the at least one surgical simulation tool ensures a colinearity constraint according to the first axis A₁ of the at least one connector.

16. The surgical training platform according to claim 11, wherein the connection system comprises a first connector attached to the haptic controller and a second connector attached to the free end of the surgical simulation tool, the two connectors being configured to cooperate with one another.

17. The surgical training platform according to claim 11, wherein the haptic controller comprises a movable arm, the movable arm having a free end intended to cooperate with the connection system.

18. The surgical training platform according to claim 11, wherein the connection system makes it possible to pass electric current between the haptic controller and the connected surgical simulation tool, in such a way as to supply the surgical simulation tool with power.

19. The surgical training platform according to claim 11, wherein the connection system makes it possible to pass electric current between the control system and the connected surgical simulation tool, in such a way as to supply the surgical simulation tool with power.

20. The surgical training platform according to claim 11, wherein each connector comprises a magnet in such a way that the connection is magnetised.
